(19) United States
(12) Patent Application Publication
(10) Pub. No.: US 2022/0110637 A1
(43) Pub. Date: Apr. 14, 2022

(54) FIXING DEVICE FOR A TIBIA ALIGNMENT SYSTEM

(71) Applicant: Aesculap AG, Tuttlingen (DE)

(72) Inventor: Roland Boettiger, Rietheim-Weilheim (DE)

(21) Appl. No.: 17/425,771

(22) PCT Filed: Jan. 30, 2020
(86) PCT No.: PCT/EP2020/052324

§ 371 Date: Jul. 26, 2021

(30) Foreign Application Priority Data
Feb. 27, 2019 (DE) 10 2019 104 965.2

(51) Int. Cl. A61B 17/15; A61B 17/90; A61B 17/92

(52) U.S. Cl. CPC A61B 17/157; A61B 2017/922; A61B 17/92; A61B 17/90

(57) ABSTRACT

A fixing device for a tibia alignment system for knee operations includes a transverse bar, the front end of which can be connected to a main rod of the tibia alignment system. The main rod is configured for an alignment along the tibia. A rear end of the transverse bar has an anchor system with at least one anchor pin that extends obliquely to the transverse bar and can be driven into a knee joint-side end of the tibia. The anchor system has a drive-in anvil into which the anchor pin is integrated and which is designed to transmit impact forces acting on the drive-in anvil from the outside to the anchor pin in order to drive the anchor pin into the tibia. The anchor pin and drive-in anvil are guided in the transverse bar in a longitudinally movable manner.

### CROSS-REFERENCE TO RELATED APPLICATION(S)

This application is the United States national phase entry of International Application No. PCT/EP2020/052324, filed Jan. 30, 2020, and claims the benefit of priority of German Application No. 10 2019 104 965.2, filed Feb. 27, 2019. The contents of International Application No. PCT/EP2020/052324 and German Application No. 10 2019 104 965.2 are incorporated by reference herein in their entireties.

### FIELD

The present invention relates to a fixing device for a tibia alignment system for knee operations comprising a transverse bar the front end of which can be connected to a main rod of the tibia alignment system, said main rod being provided for an alignment along a tibia, and a rear end of which has an anchor system with at least one anchor pin that runs obliquely, in particular transversely, to the transverse bar and can be driven into a knee joint-side end of the tibia.

### BACKGROUND

For knee operations, at first a tibia alignment system such as an extra-medullary tibia alignment instrument (ETA) is fastened via a foot-side fixation and a knee-side fixation so that a main rod (adjusting rod) is aligned along a shin or a tibia of a patient. Within the scope of the application, said alignment is referred to as vertical, which is merely intended to facilitate the description and does not refer to an alignment in space. Equally, in the following the term “front” is used as facing the main rod of the tibia alignment system and the term “rear” is used as turned away therefrom. After that, a saw guide or saw jig connected or retained to the main rod can be positioned and aligned at the knee via the tibia alignment system. This means that the tibia alignment system is connected to the tibia at the bottom (i.e., along the main rod at a foot-side end thereof) for example by a foot clamp (foot-side fixation) and at the top (i.e., along the main rod at a knee-side end thereof) via the transverse bar as a C-bend. For adjusting the cutting depth, the saw jig/saw guide is then displaced, respectively, adjusted relative to the entire support structure (C-bend) in the vertical direction or in parallel to the tibia or along the main rod. This can be done via a grid or a thread adjustment. To perform a cut by means of the saw guide or saw jig, the latter must be fastened to the tibia and subsequently the tibia alignment system must be removed again.

The tibia alignment system can be fixed on the knee side in substantially two ways, as is known. On the one hand, starting from the front (i.e., from anterior or starting from a side facing a main rod of the tibia alignment system) a pin can be pushed through a mounting hole in the applied saw jig or saw guide. In order to be able to subsequently set a cutting depth, the mounting hole must be designed as a elongated hole. Inside said elongated hole, the cutting depth then can be set via detents or via a threaded nut or a clamping screw in the vertical direction or along the main rod or in the direction of the cutting depth. On the other hand, the knee-side fixation can be carried out by means of a transverse bar from above (proximal) in a front face or in an eminence of the tibia on the knee joint. For this purpose, for example two pins project from the transverse bar which is connected to the vertical main rod of the tibia alignment system. The transverse bar, respectively, the projecting pins at the transverse bar are driven into the eminence. As an alternative, pins can also be screwed or driven through the transverse bar.

It is a problem of the afore-described solutions to introduce the fixing pins. If screwing pins are used, there is a risk of too long pins being used which may injure blood vessels when they are driven, especially motor-driven, into the tibia. In order to avoid the risk of too long pins, typically the transverse bar with the projecting pins of predetermined length is used, respectively, driven in.

After fixing the tibia alignment system, as already afore-described, the cutting depth is set and the saw jig is fixed to the tibia. For carrying out a tibial cut, the upper/frontal (proximal) pins must be removed from the eminence again, however. This is another problem: Since finally the saw jig/saw guide is adjustably connected (via a grid or thread adjustment) to the entire support structure, this connection must be released when the transverse bar, respectively, the projecting pins thereof are withdrawn from the eminence. Otherwise, the already fixed saw jig/saw guide is pulled. In the worst case, the connection is released simultaneously with the removal of the transverse bar.

For example, from U.S. Pat. No. 7,344,542 B2 a tibial alignment guide is known in which the transverse bar is rigidly connected to the main rod and includes a lever which, upon actuation, presses against the tibia to withdraw the pins arranged on the transverse bar from the tibia. This means that the entire transverse bar with the integrated pins is levered out against the bone, with the connection of the saw jig to the vertical main rod having to be simultaneously released in an adverse manner.

Further, from US 2010/0087831 A1 a surgical fixation pin remover is known which can grip under a fixation pin head and extract the same from the bone by means of a lever and a gear.

Furthermore, U.S. Pat. Nos. 6,090,114 A and 7,344,542 B2 disclose a fixing device for a tibia alignment system for knee operations. The fixing device includes a transverse bar the front end of which can be connected to a main rod of the tibia alignment system provided for an alignment along a tibia, and a rear end of which includes an anchor system with at least one anchor pin that extends obliquely to the transverse bar and can be driven into a knee joint-side end of the tibia.

### SUMMARY

Therefore, it is the object of the invention to avoid or at least reduce the drawbacks of the state of the art and, in particular, to provide a fixing device for a tibia alignment system, particularly an upper/frontal fixation which can be easily handled both when it is attached and when it is removed.

The object underlying the invention is achieved by a fixing device for a tibia alignment system for knee operations, comprising a transverse bar the front end of which can be connected to a main rod of the tibia alignment system, said main rod being provided for an alignment along the tibia, and the rear end of which has an anchor system with at least one anchor pin that extends obliquely to the transverse bar and can be driven into a knee joint-side end of the tibia. The anchor system further includes a drive-in anvil into which the at least one anchor pin is integrated and which is designed to transmit impact forces acting on the drive-in anvil from the outside to the at least one anchor pin in order to drive the same into the tibia, said anchor pin and said drive-in anvil being guided/held in the transverse bar in a longitudinally movable manner for this purpose.

In other words, a fixing device having a transverse bar is provided in which a drive-in anvil with pins integrated therein for driving the pins into a shin/tibia is supported/held such that the pins can be driven into the tibia by an external force acting on the drive-in anvil without moving or displacing the transverse bar. Accordingly, forces can be prevented from being transmitted from the anchor system when releasing the same via the transverse bar to the main rod and, in so doing, from possibly displacing the saw jig or saw guide. The drive-in anvil may have different shapes such as a cuboid shape or a cylindrical shape. Preferably, it moves in the direction of the pins upward from the transverse bar or away from the knee to be operated such that it protrudes from the remaining components of the fixing device and possibly during an operation from a femoral bone so that an upper end (i.e., remote from the tibia during an operation) of the drive-in anvil can be easily reached or accessed by a hammer or comparable instruments.

Accordingly, advantageously the knee-side fixation of the tibia alignment system can be produced and/or released by extracting the drive-in anvil and the at least one anchor pin upwards from the tibia and at least partially from the transverse bar without moving a main rod of the tibia alignment system on which a saw jig/saw guide is mounted. Correspondingly, easy, comfortable as well as safe handling of the fixing device according to the invention is possible. In particular, the holding fixture/guide of the at least one anchor pin or of the drive-in anvil is further configured so that they are captively held, respectively, movably connected to the transverse bar.

Preferably, a drive-in length of the at least one anchor pin is defined by a stop between the drive-in anvil and the transverse bar. For example, a front face or a step of the drive-in anvil can act as a stop. Thus, the anchor pin can advantageously be prevented from being driven too deeply into the tibia, thereby avoiding corresponding injuries of blood vessels, for example.

Preferably, the fixing device further comprises an auxiliary device for releasing the drive-in anvil and of the at least one anchor pin. As auxiliary device preferably a lever is provided which is supported centrally on the transverse bar and, at one lifting end, is hinged to or supported in the at least one anchor pin or the drive-in anvil so as to displace the same relative to the transverse bar in the longitudinal direction of the pin.

In other words, the lever is preferably captively hinged either to the drive-in anvil or the at least one anchor pin and centrally bears on the transverse bar; or the lever is preferably captively hinged centrally to the transverse bar and at the lifting end bears on the drive-in anvil or the at least one anchor pin. In other words, a hinge point of the preferably captively hinged lever may either be arranged on the drive-in anvil or the at least one anchor pin, wherein a support point of the lever is located, when the latter is actuated, centrally on the transverse bar. Or, a hinge point of the preferably captively hinged lever may be arranged centrally on the transverse bar, wherein a support point of the lever arranged on the lifting end of the lever is located, when the latter is actuated, on the drive-in anvil or the at least one anchor pin.

Basically, it is also possible to provide a separately provided lever which can be clamped as a single part between the transverse bar and a base of the anchor system to lever the at least one anchor pin out of the tibia.

In this way, the anchor system with the at least one anchor pin and the drive-in anvil and the transverse bar can be forced away from each other by the lever, thereby the at least anchor pin being extracted from the tibia during an operation. This helps prevent the lever from bearing against the bone, therefore hardly any forces are transmitted to the patient and this configuration is gentle to the tissue. Further, due to the leverage effect of the lever, the operating surgeon only has to apply low forces, which is why he/she will fatigue less quickly, and moreover a smooth steady removal of the anchor pins is possible.

Preferably, the lever and the drive-in anvil are arranged on an upper side of the transverse bar as a different side relative to the at least one anchor pin. In other words, preferably both the hinge point and the support point of the lever are located on the upper side or in a region above the upper side of the transverse bar. Accordingly, particularly the lever can be positioned or positionable so that, when it is actuated, for releasing the drive-in anvil and the at least one anchor pin a moving range and/or kinematic degrees of freedom of the lever are located exclusively on an upper side of the transverse bar and, thus, not on the side of the transverse bar facing the at least one anchor pin. The term moving range especially covers a developing (or evolving) line of movement of at least one contact point between the lever and the drive-in anvil to be released with the at least one anchor pin. That is to say, upon actuation of the lever, crossing of the lever as auxiliary device for releasing the drive-in anvil and the at least one anchor pin, respectively, of a region and/or section of the lever with the transverse bar is avoided. Thus, the lever, respectively, the section of the lever is effectively prevented, when it is actuated, from moving to the vicinity of the patient at all. In this way, this preferred embodiment offers the special advantage of the risk of a, particularly painful and/or traumatizing, contact with and/or mechanical application of force to the patient, especially the tibia, by the auxiliary device, respectively, the lever being completely avoided in advance, respectively, due to the constructional design.

According to another aspect of the invention, at its front end, the transverse bar includes a guide member via which the transverse bar is supported/retained on/in the main rod of the tibia alignment system to be freely movable along the bar free from stops, especially to the front and the rear. In particular, the transverse bar is supported/retained or supportable/retainable to be freely retractable on the main bar. For example, a bearing eye in which the transverse bar is retained via a cylindrical support surface is provided on the main rod for this purpose. This enables the operating surgeon to easily insert the transverse bar of the fixing device during operation into the main rod of the tibia alignment system and to remove the same without pulling the saw jig/saw holder. Accordingly, the risk of displacing the latter when removing the tibia alignment system can be minimized. In addition, the handling of the tibia alignment system when removing and when attaching the fixing device is significantly facilitated and accelerated.

Preferably, a distance of the at least one anchor pin from the main rod can be adjusted by the support of the transverse bar movable along the bar axis on/in the main rod. In this way, after the pins are driven in, the main rod can be displaced forward while being guided by the transverse bar on the knee side, thus causing the cutting block (saw jig/saw holder) responsible for the cutting height to be spaced apart from the tibia and subsequently to be movable free from collision with the tibia upward or downward/along the main rod. In the case of a fixed distance between the at least one anchor pin and the main rod, the cutting block may possibly abut against the tuberosity of the tibia and might injure the same.

According to a preferred embodiment, two anchor pins extending in parallel to each other, in particular being offset along the transverse bar, are provided which are connected to the drive-in anvil. This helps improve fixation of the anchor system on the tibia.

In other words, the object underlying the invention is achieved by a proximal (upper/frontal) fixation (extramedullary tibia alignment instrument—ETA) which (as fixing device) comprises a multi-part transverse bar (with separate components movable relative to each other) that can be inserted, after applying the ETA, from the side of the operating surgeon. The transverse bar remains movable in the anterior-posterior direction in the ETA (displaceable to the front and the rear) so that it can adjust to the respective size and depth of the tibia plateau. The fixation to the tibia itself is carried out by means of at least one pin, preferably two pins, which are integrated in an anvil/drive-in anvil. The anvil is designed so that it protrudes from the flexed femur and can be driven into the eminence by a hammer. For removing the pins, a lever which levers out the anvil with the pins is moved downward in the direction of the transverse bar. Subsequently, the multi-part transverse bar can be extracted from the ETA toward the operating surgeon. The saw jig position is not affected. All component parts of the transverse bar are preferably captively interconnected. In particular, the integrated drive-in anvil with the pins is relevant. The transverse bar is not connected tightly to the ETA but can be moved to the front or to the rear out of a guide and can adjust to the depth of the tibia plateau. The transverse bar also includes a lever for extracting the pins which are integrated in the drive-in anvil.

### BRIEF DESCRIPTION OF THE DRAWING FIGURES

In the following, the present invention is described by way of an example embodiment with the aid of the attached Figures. The Figures are not intended to limit the scope of the present invention but merely serve for illustration purposes.

FIG. 1 shows a side view of a fixing device according to the invention.

FIG. 2 shows a rear view of the fixing device of FIG. 1.

FIG. 3 shows a side view of the fixing device during an operation.

### DETAILED DESCRIPTION

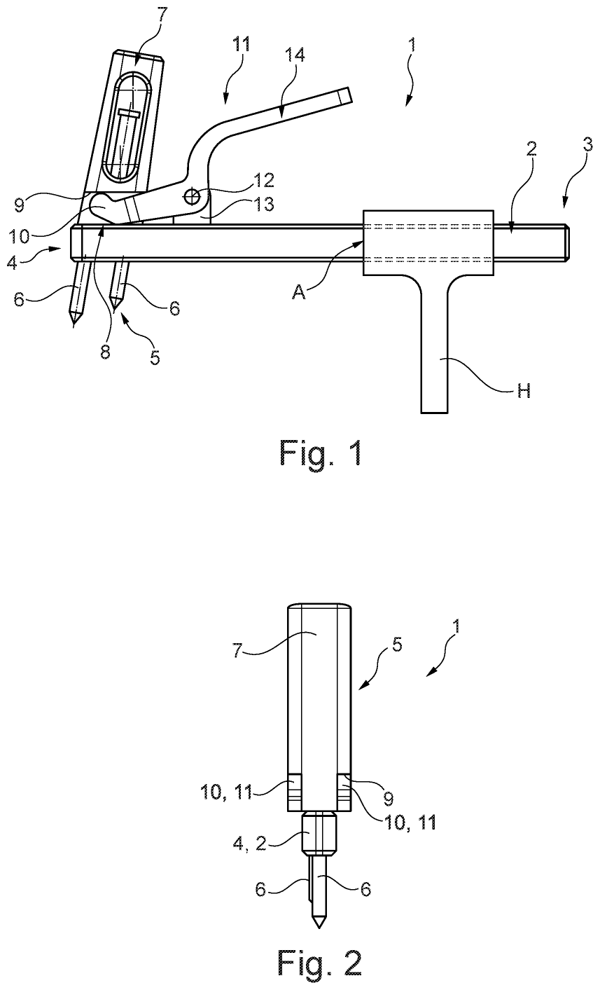

The fixing device 1 shown in FIG. 1 comprises a transverse bar 2 at the front end 3 of which (on the right in FIG. 1) a preferably cylindrical guide is provided through which the transverse bar 2 can be inserted into a corresponding seat A of the main rod H of a tibia alignment system (ETA). At a rear end 4 (on the left in FIG. 1) of the transverse bar 2 an anchor system 5 is provided which includes two anchor pins 6 arranged to be axially spaced in the longitudinal direction of the bar, said anchor pins 6 protruding obliquely, preferably at an angle of 80° to 130°, from the lower side (viewed in FIG. 1) of the transverse bar 2.

At the top/on a far side of the tibia T (at the top in FIG. 1), the anchor pins 6 are connected to/held on/integrated in/fastened to a drive-in anvil 7 that is disposed on an upper side of the transverse bar 2 and extends upward in the longitudinal direction of the pin. Further, the anchor pins 6 are of different length, in particular the more rearward anchor pin 6 is shorter than the anchor pin 6 at the front.

A lower front face of the drive-in anvil 7 facing the tibia T and the transverse bar is adapted, particularly beveled, so as to bear on the upper side of the transverse bar 2 in a driven-in position of the anchor system 4 shown here and, thus, to provide a stop 8 for restricting a drive-in length of the anchor pins 6. The drive-in anvil 7 further forms, at its lower end (at the bottom in FIG. 1) a step 9 onto which a lifting end 10 of a lever 11 engages so as to press the anchor system 4 away from the transverse bar 2. For this purpose, the rounded lifting end 10 presses against the step 9. Therefore, the lever 11 acts as an auxiliary device for releasing the drive-in anvil 7 with the two anchor pins 6, 6.

The lever 11 is S-shaped and extends substantially forward. At a central area of the S-shape of the lever 11, a hinge pin 12 is provided which is inserted in corresponding hinge eyes for forming a hinge point that is configured in an extension/projection 13 on the upper side of the transverse bar 2. The hinge pin 12 is perpendicular but offset to the longitudinal axis of the transverse bar 2.

As can be seen from FIG. 1, the lever 11 and the drive-in anvil 7 are arranged on the upper side of the transverse bar 2 so that they are located on a different (other) side of the (than the) two anchor pins 6, 6 (i.e., far side of the tibia). Thus, both the hinge point of the lever 11 formed by the hinge pin 12 in the corresponding hinge eyes and a support point of the lever 11 formed between the lifting end 10 and the step 9 are provided on the upper side of the transverse bar 2.

A handle end 14 of the lever 11 opposed to the lifting end 10 serves as a handle to actuate the lever. The S-shape of the lever 11 ensures sufficient space to be provided between the handle end 14 and the upper side of the transverse bar 2 so that the handle end 14 can be grasped and moved. Upon actuation of the lever 11 by pressing down the handle end 14 toward the upper side of the transverse bar 2, the support point lifts rocker-like in the direction away from the upper side of the transverse bar 2. Accordingly, the line of movement of the support point will not cross the longitudinal axis of the transverse bar 2. Thus, the actuation, respectively, the kinematic course of movement of the lever 11 takes place on the side of the transverse bar 2 far from the two anchor pins 6, 6, i.e. far from the tibia, and in a space above the upper side of the transverse bar 2.

FIG. 2 illustrates a rear view of the afore-described fixing device 1. It is evident from FIG. 2 that the drive-in anvil 7, the lever 11, the transverse bar 2 and the anchor pins 6 are arranged in a line symmetrically to each other, respectively, symmetrically to a plane of symmetry that extends in the vertical direction, as viewed in FIG. 2. In particular, the bifurcated lifting end 10 is shown which, in the area of the step 9, encompasses the drive-in anvil 7 to press upward against the step 9. At its front face 8, the drive-in anvil 7 abuts on the rear end 4 of the transverse bar 2 to restrict the drive-in length of the anchor pins 6. Further, the transverse bar 2 is sufficiently narrow at a rear end so that it can be located between the tines/jaws of the bifurcated lifting end 10. Thus, easy handling and mounting of the lever 11 is ensured.

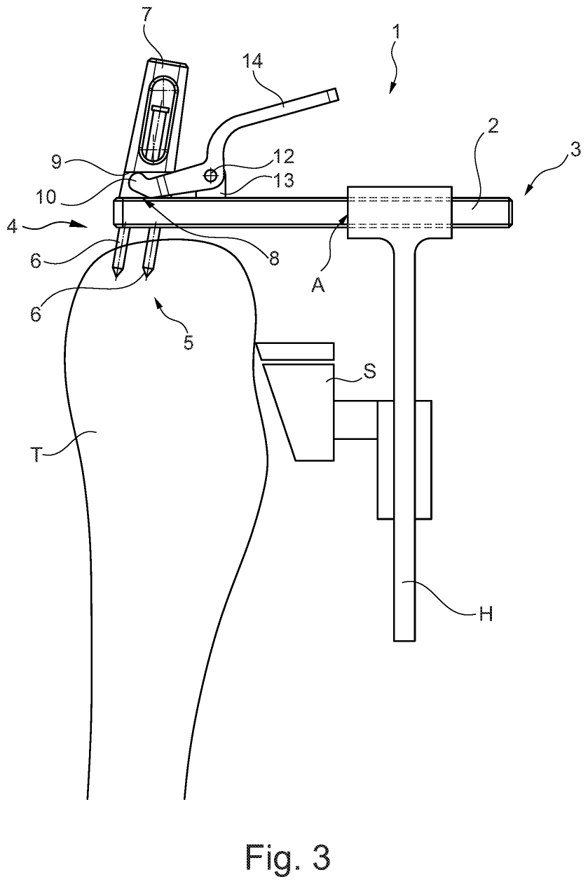

The view shown in FIG. 3 substantially corresponds to a simplified representation of the embodiment of the fixing device 1 according to the invention of FIG. 1, but it illustrates the fixing device 1 when it is utilized in a knee operation. It can be seen in more detail that the anchor pins 6 which are retained at the rear end 4 of the transverse bar 2 are driven from above (as seen in FIG. 3) or frontally into an upper front face of the tibia T. The transverse bar 2 is thus fixed to the tibia T. The transverse bar extends forward in the direction of the main rod H and is guided on the latter movably along the bar axis.

On the main rod H extending substantially in parallel to the tibia T, a cutting block S, respectively, a saw jig or saw holder is held to be movable along the main rod H. If the cutting block S is to be displaced downward, with a rigid connection of the main rod H and the transverse bar 2, the cutting block would abut against or rub along and damage a part of the tibia T, as is clearly visible in this schematic view. As, however, the main rod H or at least the upper end thereof is movable forward along the fixed transverse bar 2, it is possible to space apart to this one jointly with the cutting block S from the tibia T. Subsequently, the cutting block S is movable (slidable) along the main rod H without any risk of injuring the tibia T.

In order to extract, after fixing the cutting block S, the pins from the front face of the tibia T, the lever 14 can be pressed down to pull the drive-in anvil 7 and the anchor pins 6 arranged/held thereon upwards relative to the tibia T and the transverse bar 2. As the anchor pins 6 are movable (slidable) along the pin axis in the transverse bar 2, the transverse bar 2 and the main rod H can remain fixed, as described in detail before, and the connection of the cutting block S/the saw jig to the main rod H need not be simultaneously released.

1. A fixing device for a tibia alignment system for knee operations, the fixing device comprising:
a transverse bar comprising a front end that is connectable to a main rod of the tibia alignment system, said main rod being provided for alignment along a tibia, the transverse bar further comprising a rear end having an anchor system with at least one anchor pin that extends obliquely to the transverse bar and is drivable into a knee joint-side end of the tibia,
the anchor system comprising a drive-in anvil for improving fixation of the anchor system to the tibia, the at least one anchor pin being connected to and integrated into the drive-in anvil, the anchor system configured to transmit impact forces acting on the drive-in anvil from outside to the at least one anchor pin in order to drive the at least one anchor pin into the tibia,
the at least one anchor pin and drive-in anvil being guided in the transverse bar in a longitudinally movable manner.

2. The fixing device according to claim 1, wherein a drive-in length of the at least one anchor pin is defined by a stop between the drive-in anvil and the transverse bar.

3. The fixing device according to claim 1, wherein said fixing device further comprises an auxiliary device for releasing the drive-in anvil and the at least one anchor pin.

4. The fixing device according to claim 3, wherein the auxiliary device comprises a lever which is mounted or supported centrally on the transverse bar, is hinged, at a lifting end, to the at least one anchor pin or the drive-in anvil or supported therein so as to displace the drive-in anvil relative to the transverse bar in a longitudinal direction of the at least one anchor pin.

5. The fixing device according to claim 3, wherein the auxiliary device comprises a separately provided lever configured to be clamped as single component part between the transverse bar and a base of the anchor system to lever the at least one anchor pin out of the tibia.

6. The fixing device according to claim 4, wherein the lever is hinged to the drive-in anvil or the at least one anchor pin and is supported centrally on the transverse bar.

7. The fixing device according to claim 4, wherein the lever is hinged centrally to the transverse bar and is supported at the lifting end on the drive-in anvil or the at least one anchor pin.

8. The fixing device according to claim 7, wherein the lever and the drive-in anvil are arranged on an upper side of the transverse bar as a different side relative to the at least one anchor pin.

9. The fixing device according to claim 8, wherein:
a lower front face of the drive-in anvil facing the tibia and/or the transverse bar is adapted to abut, in a driven-in position of the anchor system, on the upper side of the transverse bar to provide a stop for restricting a drive-in length of the at least one anchor pin;
the drive-in anvil forms a step at its lower end for engaging the lifting end of the lever to force the anchor system away from the transverse bar;
the lever is designed to be S-shaped and to extend substantially forward and is hinged by a hinge pin, in corresponding hinge eyes formed in an extension and/or a projection on the upper side of the transverse bar; and
a handle end of the lever opposed to the lifting end is provided for actuation of the lever.

10. The fixing device according to claim 1, wherein the transverse bar at its front end includes a guide member via which the transverse bar is supportable on the main rod of the tibia alignment system to be freely movable along an axis of the bar.

11. The fixing device according to claim 10, wherein the transverse bar is supportable on the main rod to be freely extractable.

12. The fixing device according to claim 10, wherein a distance of the at least one anchor pin from the main rod is adjustable by support of the transverse bar movable along the axis of the bar so as to adjust a cutting depth.

13. The fixing device according to claim 1, wherein the at least one anchor pin comprises two anchor pins extending in parallel to each other which are connected to the drive-in anvil.
